(19) United States
(12) Patent Application Publication
(10) Pub. No.: US 2006/0173176 A1
(43) Pub. Date: Aug. 3, 2006

(54) Process for the preparation of (z)-isomer enriched 7-amino-3-propen-1-yl-3-cephem-4-carboxylic acid

(76) Inventors: Yatendra Kumar, Haryana (IN); Neera Tewari, Haryana (IN); Shailendra Kumar Singh, Haryana (IN); Bishwa Prakash Rai, Uttar Pradesh (IN)

Correspondence Address: RANBAXY INC., 600 COLLEGE ROAD EAST, SUITE 2100, PRINCETON, NJ, 08540, US

(21) Appl. No.: 10/530,589

(22) PCT Filed: Oct. 8, 2003
(86) PCT No.: PCT/IB03/04439

§ 371 Date: Nov. 17, 2005

(30) Foreign Application Priority Data
Oct. 8, 2002 (IN) 1024/DEL/2002

(51) Int. Cl. C07D 501/00

(52) U.S. Cl. 540/230

(57) ABSTRACT

The invention relates to a process for enrichment of the (Z)-isomer amount in a mixture of the (Z)- and (E)-isomers of 7-amino-3-(1-propen-1-yl)-3-cephem-4-carboxylic acid.

### FIELD OF THE INVENTION

The invention relates to a process for enrichment of the (Z)-isomer component in a mixture of the (Z)- and (E)-isomers of 7-amino-3-(1-propen-1-yl)-3-cephem-4-carboxylic acid of Formula I

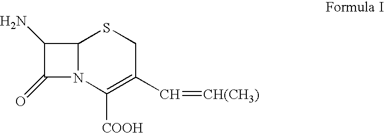

### BACKGROUND OF THE INVENTION

The compound of Formula I is an important intermediate for the preparation of 3-propenyl cephalosporin antibiotics such as cefprozil and BAYv 3522. Synthetic processes for the production of these antibiotics generally yield mixtures containing both the (Z)- and (E)-isomers. The Z-configuration of the propenyl group is related to the activity of 3-propenyl cephalosporin antibiotics against the gram negative bacteria, hence, the need to minimize the undesired (E)-isomer in these antibiotics.

U.S. Pat. No. 4,727,070 describes a process for preparing cefprozil that is substantially free of the corresponding E-isomer. The process involves preparation of the sodium salt of imidazolidinone derivative of a mixture containing cefprozil and its corresponding E-isomer, and separation of the imidazolidinone derivative isomers based on their differential solubility.

U.S. Pat. No. 6,136,967 describes a process for preparing (Z)-isomer enriched 7-amino-3-(1-propen-1-yl)-3-cephem-4-carboxylic acid of Formula I involving depleting the corresponding (E)-isomer in a mixture of the (Z)- and (E)-isomers of carboxylic acid of Formula I by subjecting a solution of the mixture to adsorption chromatography.

U.S. Pat. No. 5,869,648 describes a process for preparing a (Z)-isomer enriched carboxylic acid of Formula I by: (1) reacting a mixture of (Z)- and (E)-isomers with a lithium, sodium or potassium base, ammonia or an amine to form a mixture of the (Z)- and (E)-isomers of the corresponding salts, (2) depleting the (E)-isomer salt from (Z)-isomer salt in a solvent or solvent mixture in which the two isomers have different solubility to recover the enriched (Z)-isomer salt of carboxylic acid of Formula I, and (3) converting it to the free acid.

U.S. Pat. No. 6,333,049 gives another variant of the above process based on the differential solubility of the (Z)- and (E)-isomers of the hydrochloride salt of the carboxylic acid of Formula I.

Cephalosporanic acid derivatives with a (cyclo)alkylideneammonio group are provided in U.S. Pat. No. 5,359,058, and are used as a method of protecting an amino group in a synthesis in which amino carboxylic acids have to be protected. Cephalosporanic acid derivatives with an aldimine substituent at the 7-position have been described for instance by W. A. Spitzer, T. Goodson, R. J. Smithey and I. G. Wright, J. C. Soc. Chem. Comm., 1338 (1972).

### SUMMARY OF THE INVENTION

In one general aspect there is provided a process for preparing (Z)-isomer-enriched 7-amino-3-(1-propen-1-yl)-3-cephem-4-carboxylic acid of Formula I, 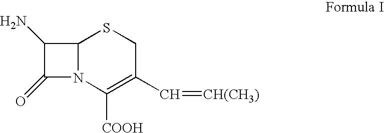
the process including:
a) reacting a mixture of the (Z)- and (E)-isomers of carboxylic acid of Formula I with a compound of Formula II 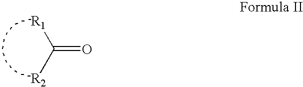
wherein R₁ and R₂ are independently hydrogen, alkyl, alicyclic, aryl, aralkyl, or R₁ and R₂ together form a 5- to 7-membered carbocyclic ring, in the presence of an acid, HX, to form a reaction mixture comprising an alkylidene ammonio salt derivative of Formula III, 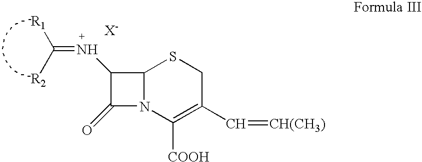
wherein R₁ and R₂ are the same as above and X is an anion from the acid HX;
b) obtaining (Z)-isomer-enriched alkylidene ammonio salt derivative of Formula III from the reaction mixture; and
c) converting the (Z)-isomer-enriched alkylidene ammonio salt derivative of Formula III to 7-amino-3-(1-propen-1-yl)-3-cephem-4-carboxylic acid of Formula I, as the free acid or in salt forms.

Embodiments of the process may include one or more of the following features. For example, the compound of Formula II may be a ketone. The ketone may be selected from one or more of acetone, methyl isobutyl ketone, cyclohexanone, cyclopentanone, and benzophenone. The compound of Formula II may be an aldehyde. The aldehyde may be selected from one or more of benzaldehyde, acetaldehyde and formaldehyde.

The acid may be an inorganic acid. The inorganic acid may be one or more of hydrogen chloride, hydrogen bromide, hydrogen iodide, sulfuric acid and perchloric acid.

The acid may be an organic acid. The organic acid may be selected from one or more of formic acid and acetic acid.

The reaction may be performed in an inert non-aqueous organic solvent or solvent mixture in which the (Z)- and (E)-isomers of the derivative of Formula III have different solubilities. The organic solvent or mixture may be such that the (Z)-isomer of the salt derivative of Formula III is relatively insoluble and the (E)-isomer is soluble. The organic solvent or mixture may be one or more of carboxylic acids, amides, sulfoxides, sulfones, halogenated hydrocarbons, ketones, esters, ethers, and nitriles. The organic solvent or mixture may be one or more of acetic acid, dimethylformamide, dimethylsulfoxide, sulfolane, dichloromethane, acetone, ethyl acetate, tetrahydrofuran, and acetonitrile.

The reaction mixture of step (a) may be diluted with a counter solvent or a mixture of counter solvents to obtain the (Z)-isomer enriched derivative of Formula III. The organic counter solvent may be one or more of ketones, ethers, esters, and nitriles. The organic counter solvent may be one or more of acetone, tertiary butyl methyl ether, diethylether, tetrahydrofuran, ethyl acetate, isopropyl acetate, and acetonitrile.

The reaction of step (a) may be performed at a temperature of between about 20° C. to about 55° C. and, more particularly, the reaction may be performed at a temperature of between about 30° C. to about 45° C.

Obtaining the (Z)-isomer-enriched alkylidene ammonio salt derivative of Formula III may include crystallizing the derivative of Formula III at a temperature of between about 0° C. to about 30° C. and, more particularly, at a temperature of between about 0° C. to about 15° C.

The conversion of the carboxylic acid of Formula I may provide the compound of Formula I having Z/E isomers in a ratio of about 91:9 to about 99:1.

The process may further include converting the (Z)-isomer-enriched carboxylic acid of Formula I to a 3-propenyl cephalosporin antibiotic. The process may further include converting the (Z)-isomer-enriched carboxylic acid of Formula I to cefprozil. The cefprozil may have Z/E isomers in a ratio of from about 91:9 to about 99:1.

The process may further include obtaining cefprozil by (1) silylating the (Z)-isomer enriched 7-amino-3-(1-propen-1-yl)-3-cephem-4-carboxylic acid of Formula I; and (2) reacting the silylated product with a mixed carboxylic acid anhydride produced by reacting a Dane salt with ethyl chloroformate.

In another general aspect, there is provided a drug product that includes a 3-propenyl cephalosporin antibiotic formed by converting the (Z)-isomer-enriched carboxylic acid of Formula I to a 3-propenyl cephalosporin antibiotic In another general aspect, there is provided a drug product that includes cefprozil formed by a process that includes converting the (Z)-isomer-enriched carboxylic acid of Formula I to cefprozil.

In another general aspect there is provided a drug product that contains cefprozil that includes Z/E isomers in a ratio of from about 91:9 to about 99:1.

In another general aspect there is provided a drug product that contains cefprozil formed by (a) silylating the (Z)-isomer enriched 7-amino-3-(1-propen-1-yl)-3-cephem-4-carboxylic acid of Formula I; and (b) reacting the silylated product with a mixed carboxylic acid anhydride produced by reacting a Dane salt with ethyl chloroformate.

In another general aspect there is provided a method of treating a condition for which an antibiotic is indicated. The method includes providing a drug product that includes a 3-propenyl cephalosporin antibiotic formed by converting the (Z)-isomer-enriched carboxylic acid of Formula I to a 3-propenyl cephalosporin antibiotic.

Embodiments of the method of treating may include any one of the features described above. For example, the 3-propenyl cephalosporin antibiotic may be cefprozil.

The details of one or more embodiments of the inventions are set forth in the description below. Other features, objects and advantages of the inventions will be apparent from the description and claims.

### DETAILED DESCRIPTION OF THE INVENTION

The prior art compounds described above were useful as synthetic intermediates. However, there has been no application of such derivatives to the separation of mixtures of cephalosporins where geometric isomerism about a double bond exists.

The inventors have developed a process for preparing the (Z)-isomer enriched 7-amino-3-(1-propen-1-yl)-3-cephem-4-carboxylic acid of Formula I. The process includes:
(i) reacting a mixture of the (Z)- and (E)-isomers of carboxylic acid of Formula I with a compound of Formula II, 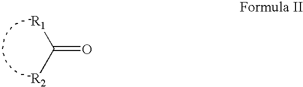
wherein R₁ and R₂ are independently hydrogen, alkyl, alicyclic, aryl, aralkyl, or R₁ and R₂ together form a 5 to 7 membered carbocyclic ring; in the presence of an acid, HX to form an alkylidene ammonio salt derivative of Formula III, 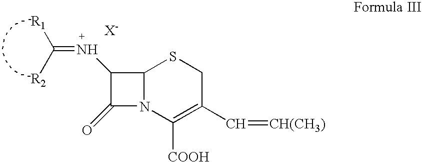
wherein R₁ and R₂ are the same as above and X is an anion from the acid HX,
(ii) obtaining (Z)-isomer enriched alkylidene ammonio salt derivative of Formula III from the above reaction mixture, and
(iii) converting the (Z)-isomer enriched alkylidene ammonio salt of Formula III to 7-amino-3-(1-propen-1-yl)-3-cephem-4-carboxylic acid of Formula I, which is obtained as the free acid or in salt form.

The free acid or a salt of the mixture of the (Z)- and (E)-isomers of the compound of Formula I may be used as the starting compound in the reaction and may have up to 30% of the (E)-isomer.

The alkyl group may be a C₁₋₆ straight or branched chain alkyl. The alicyclic group may be a 5 to 7 membered carbocyclic group. The aryl group may be phenyl, which may be further substituted by alkyl, halogen, alkoxy or hydroxy groups.

The compound of Formula II may be a ketone such as acetone, methyl isobutyl ketone, cyclohexanone, cyclopentanone, or benzophenone; or an aldehyde such as benzaldehyde, acetaldehyde or formaldehyde.

The acid may be any suitable inorganic or organic acid. The acid is typically added as a concentrated anhydrous solution or purged into the reaction mixture in the gaseous form. Suitable inorganic acids include hydrogen chloride, hydrogen bromide, hydrogen iodide, sulfuric acid and perchloric acid. Suitable organic acids include formic acid and acetic acid.

Thus, X⁻ in the salt derivative of Formula III may be Cl⁻, Br⁻, I⁻, ClO₄⁻, HSO₄⁻, HCOO⁻ or CH₃COO⁻.

The acid and the aldehyde or ketone of Formula II used in the reaction may also act as solvents for the reaction. In addition, a suitable organic solvent may also be employed. Where the aldehyde or ketone used is not a suitable solvent material, the aldehyde or ketone may be provided as a solute in an organic solvent. The solvent may be any reaction-inert, non-aqueous organic solvent or solvent mixture in which the (Z)- and (E)-isomers of alkylidene ammonio salt derivative of Formula III have different solubilities.

A solvent is selected in which the (Z)-isomer of the salt derivative of Formula mi is relatively insoluble, while the (E)-isomer is soluble. Testing of various combinations of aldehydes/ketones, acids, and solvents to accomplish this purpose is within the skill of the laboratory chemist.

Operating in a practically water-free system, the mixture of the (Z)- and (E)-isomers of carboxylic acid of Formula I is dissolved or suspended in an acid, and then a compound of Formula II and optionally an organic solvent is/are then added. Once the salt derivative of Formula III is formed, the reaction mixture is optionally diluted with a counter solvent or a mixture of counter solvents, whereby the crystalline (Z)-isomer enriched derivative of Formula III is crystallized out. Selective precipitation of the (Z)-isomer of the salt derivative of Formula III occurs due to the lower solubility thereof, relative to the (E)-isomer derivative. The crystalline (Z)-isomer enriched derivative of Formula III is recovered by filtration or centrifugation.

Examples of organic solvents are carboxylic acids, e.g., acetic acid; amides, e.g., dimethylformamide; sulfoxide, e.g., dimethylsulfoxide; sulfone, e.g., sulfolane; halogenated hydrocarbons, e.g., dichloromethane; ketones, e.g., acetone; esters, e.g., ethyl acetate; ethers, e.g., tetrahydrofuran; nitriles, e.g., acetonitrile or mixtures of these solvents. Further solvents may be added in admixture, such as diethyl ether or tertiary butyl methyl ether.

Suitable organic counter solvents are, in particular, ketones, e.g., acetone; ethers, e.g., tertiary butyl methyl ether, diethylether, tetrahydrofuran; esters, e.g., ethyl acetate, isopropyl acetate; nitriles, e.g., acetonitrile; or mixtures thereof.

The reaction may be performed at room temperature or at a somewhat elevated temperature, such as a temperature of about 20° C. to about 55° C., or at a temperature of about 0° C. to about 45° C. The product of Formula III is crystallized out at room temperature or at a lower temperature, such as a temperature of about 0° C. to about 30° C., or at a temperature of about 0° C. to about 15° C.

According to another variant, the derivative of Formula III obtained from the reaction may be suspended or dissolved in a solvent or solvent mixture in which the (E)-isomer of Formula III is more soluble than the corresponding (Z)-isomer. Suitable solvents are organic solvents mentioned above. Precipitation is then induced by, e.g., adjusting the solubility product of the (Z)- or (E)-isomer by optional addition of one of the above mentioned counter-solvents, whereby the derivative of Formula III, with a reduced (E)-amount, is obtained.

The derivative of Formula III, which is thereby much improved in its Z/E ratio may subsequently be converted again into the carboxylic acid of Formula I in conventional manner, e.g., by means of pH adjustment in water to the approximate isoelectric point.

Compounds of Formula I containing various amounts of Z/E isomers, from a ratio of about 91:9 to about 99:1 or more may be prepared with good yields and purity, as described by the processes herein. The process may be repeated in order to obtain the desired Z/E ratio.

The crystalline alkylidene ammonio salt derivatives of Formula III are new and also form part of the invention. The derivatives of Formula III having a Z/E ratio of at least 91:9 or more are also new and form part of the invention. These compounds are useful as intermediates in the process for the preparation of (Z)-isomer enriched 7-amino-3-(1-propen-1-yl)-3-cephem-4-carboxylic acid of Formula I.

The (Z)-isomer enriched carboxylic acid of Formula I is converted to a 3-propenyl cephalosporin antibiotic by methods known in the art, such as those described in U.S. Pat. Nos. 4,699,979; 5,171,854; 5,608,055; and 6,060,268, and U.S. patent application Ser. No. 2002/120,136, which are incorporated herein by reference.

In particular, cefprozil may be prepared by a process that includes:
i) producing a mixed carboxylic acid anhydride by reacting a Dane salt with ethyl chloroformate, and
ii) reacting the obtained mixed carboxylic acid anhydride with a silylated (Z)-isomer enriched 7-amino-ceph-3-em-4-carboxylic acid of Formula I obtained by the process of the present invention, to obtain cefprozil in good yield and purity.

The Dane salt may be selected from sodium or potassium (D)-N-(1-methoxycarbonyl-propen-2-yl)-α-amino-p-hydroxyphenylacetate and sodium or potassium (D)-N-(1-ethoxycarbonylpropen-2-yl)-α-amino-p-hydroxyphenylacetate.

A base, e.g., a tertiary amine base such as N-methyl morpholine, N,N-dimethyl benzyl amine, triethylamine, pyridine, picoline, or lutidine is used as a catalyst for mixed carboxylic acid anhydride formation.

The mixed anhydride may be prepared in a solvent conventionally used, such as a halogenated hydrocarbon, e.g., methylene chloride; a ketone, e.g., methyl isobutyl ketone; an ester, e.g., ethyl acetate; or an aromatic hydrocarbon, e.g., toluene; and a co-solvent such as an organic amide. Organic amide is selected from formamide, acetamide, N,N-dimethyl formamide, N-methylacetamide, N,N-dimethylacetamide and N-methylpyrrolidone.

The solvents used for mixed anhydride preparation may also be used for the condensation of step ii).

The antibiotic, cefprozil, containing various amounts of Z/E isomers, from a ratio of about 91:9 to about 99:1 or more may be prepared according to the processes described herein.

In the following section preferred embodiments are described by way of examples to illustrate the processes of the invention. However, these are not intended to limit the scope of the present invention. Multiple variants of these examples would be evident to one of ordinary skill in the art.

### Preparation of (6R,7R)-7-isopropylideneammonio-3-[(Z/E)-1-propen-1-yl]-3-cephem-4-carboxylic acid hydrochloride

### EXAMPLE 1

Hydrogen chloride gas (100 g) was passed through a mixture of acetic acid (200 ml) and acetone (500 ml) at a temperature of between 25° C. to 35° C. 7-Amino-3-(1-propen-1-yl)-3-cephem-4-carboxylic acid (100 g, Z/E ratio: 75/25) was added at a temperature of between 30° C. to 35° C. in 2 to 3 minutes and stirred to obtain a clear solution. Acetone (500 ml) was then added in 5 minutes and the stirring continued. Solid separated from the clear solution. The reaction mixture was cooled to a temperature of 0° C. to 5° C. and stirred for 2 to 3 hours. The solid was filtered, washed with acetone, and dried to obtain 100 g of the title compound.

Z/E Ratio: 90.0/9.5, (E)-isomer content (By NMR): 9.0%, Chloride content: 12%

| | |
|---|---|
| ¹HNMR (300 MHz): | 2.54 (d, 3H, CH₃, Z-Isomer), 2.56 (d, 3H, CH₃, |
| (CF₃COOD) δ value | E-Isomer) |
| | 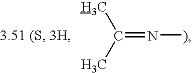 |
| | 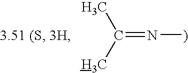 |
| | 4.25-4.55, (m, 2H, —SCH₂—), 6.3 (d, 1H, |
| | β-lactam), 6.73-6.86 (m, 2H, CH = CHCH₃ & |
| | β-lactam), 7.22-7.34 (d, 1H, CH = CHCH₃ & |
| | Z-isomer & E-Isomer) |
| IR (KBr, cm⁻¹): | 3426, 2906, 1780, 1707, 1653, 1621, 1404, 1351, |
| | 1213, 809, 718 and 691 |

### EXAMPLE 2

7-Amino-3-(1-propen-1-yl)-3-cephem-4-carboxylic acid (100 g, Z/E: 80/20) was dissolved in a mixture of acetic acid (200 ml) and acetone (500 ml) saturated with hydrogen chloride gas at a temperature of 30° C. to 35° C. After 5 minutes, acetone (500 ml) was added and a solid separated from the clear solution. After stirring at a temperature of 0° C. to 5° C. for 2 to 3 hours the title product was filtered, washed with acetone, and dried.

| Yield | 110 g |
|---|---|
| Z/E Ratio | 91.0/9.0 |
| (E)-isomer content (By NMR) | 8.5% |
| Chloride content | 14% |

Table I demonstrates the experiments carried out with variable Z/Es ratio of carboxylic acid of Formula I with respect to yield and the resulting Z/E ratio of the isopropylidene ammonio derivative of Formula III obtained.

TABLE I

| | | | Yield (w/w) of | |
|---|---|---|---|---|
| | Input | Input | derivative of | Achieved |
| Example No. | (Formula I) | Z/E Ratio | Formula III | Z/E |
| 3 | 100 g | 80/20 | 110 g | 92.0/8.0 |
| 4 | 100 g | 85/15 | 100 g | 92.0/8.0 |
| 5 | 100 g | 88.5/11.5 | 115 g | 93.5/6.5 |

### Regeneration of 7-amino-3-[(Z/E)-1-propen-1-yl]-3-cephem-4-carboxylic acid from 7-isopropylideneammonio-3-[(Z/E)-1-propen-1-yl]-3-cephem-4-carboxylic acid hydrochloride

### EXAMPLE 6

7-Isopropylideneammonio-3-[(Z/E)-1-propen-1-yl]-3-cephem-4-carboxylic acid hydrochloride salt (from Example I, 100 g, Z/E ratio: 90.0/9.5) was suspended in water (2,000 ml) and the pH was adjusted to 8.0-8.5 to obtain a clear solution. Activated carbon was added and stirred for 15 minutes, filtered and washed with water. The pH of the filtrate was adjusted to 3.0-3.5 with 6N hydrochloric acid. The solid so obtained was stirred for an additional 30 minutes at room temperature, filtered and then washed with water followed by acetone. Drying at 48° C. to 50° C. resulted in 75 g of the title product.

Z/E Ratio: 91.0/9.0, E-Content (By NMR): 8.9%, Assay (By HPLC): 99.5%

NMR (300 MHz, CF₃COOD): 2.47-2.50 (d, 3H, CH₃, Z-isomer), 2.66-2.68 (d, 3H, CH₃, E-isomer), 4.17-4.43 (m, 2H, SCH₂), 5.92-6.1 (m, 2H, b-lactam), 6.71 (dq, 1H, —CH═CH—CH₃), Z-isomer), 7.21-7.24 (d, 1H, CH═CH—CH₃), Z-isomer)

### EXAMPLE 7

7-Isopropylideneammonia-3-[(Z/E)-1-propen-1-yl]-3-cephem-4-carboxylic acid hydrochloride salt (100 g, Z/E: 92.0/8.0) from Example 3 (Table I) was suspended in water (2,500 ml) and dissolved by adjusting pH to 8.0 to 8.5 at room temperature. The reaction mixture was filtered and pH adjusted to 3.0-3.5 with 6N hydrochloric acid to obtain the product.

| Yield | 78 g |
|---|---|
| Z/E Ratio | 92.0/8.0 |
| (E)-isomer content (By NMR) | 7.8% |
| Assay (By HPLC) | 99.7% |

### Preparation of 7[(D)-2-amino-2-(4-hydroxyphenyl)acetamidol-3-(Z/E)-1-propenyl]-ceph-3-em-4-carboxylic acid (cefprozil) dimethylformamide solvate

### EXAMPLE 8

Solution A—To a stirred slurry of 7 amino-3-[(Z/E)-1-propen-1-yl]-cephe-3-em-4-carboxylic acid (50 g, Z/E: 92.0/8.0) in methylene chloride (300 ml) was added hexamethyldisilazane (25.2 g), trimethylchlorosilane (17.6 g), and imidazole (0.5 g). The reaction mixture was refluxed for 3.5 to 4 hours and then cooled to −10° C. to −15° C.

Solution B—Potassium (D)-N-[1-methoxycarbonyl propen-2-yl]-α-amino-p-hydroxyphenylacetate (Dane salt, 70.75 g) was stirred in methylene chloride (300 ml). The slurry was cooled to −25° C. and N,N-dimethylformamide (DMF, 400 ml) was added. The slurry was cooled to −30° C. to −35° C. and N-methyl morpholine (0.46 g) was added, followed by the addition of ethylchloroformate (8.2 g) at −35° C. This was then stirred for 1.0 hour and cooled to 45° C.

The above silylated mass (solution A) was added into the mixed anhydride (solution B) at −45° C. and stirred for 2.0-3 hours at −25° C. to −20° C. The reaction was monitored by HPLC. After completion of the reaction, a mixture of water and hydrochloric acid was added to the reaction mixture and stirred for 10 minutes. The aqueous layer was separated.

Dimethylformamide (500 ml) was added to the aqueous layer followed by activated carbon (5 g). This was stirred for 5 minutes and then the aqueous layer was filtered and washed with dimethylformamide. The pH of the aqueous phase was adjusted to 6.5 with ammonia solution at 25° C.-30° C. The white solid so obtained was filtered and washed with dimethylformamide followed by acetone. After drying at room temperature under vacuum, 98 g of cefprozil (yield: 92%, Z/E: 92.0/8.0) was obtained as dimethyl formamide solvate.

While several particular forms of the inventions have been described, it will be apparent that various modifications and combinations of the inventions detailed in the text can be made without departing from the spirit and scope of the inventions. For example, the cefprozil made by the processes described herein can be used in a drug product dosage form, e.g., tablet, capsule, sachet, dispersible tablet, solution, etc., with varying delivery characteristics, e.g., osmotic, delayed release, immediate release, sustained or extended release, modified release, etc. The dosage form also can contain active ingredients in addition to cefprozil. Moreover, the Z/E ratio of the starting material may vary beyond that disclosed herein. Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

We claim:

1. A process for the preparation of (Z)-isomer-enriched 7-amino-3-(1-propen-1-yl)-3-cephem-4-carboxylic acid of Formula I, 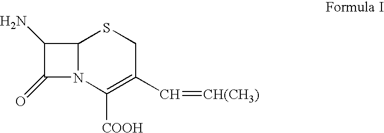
the process comprising:
(a) reacting a mixture of the (Z)- and (E)-isomers of carboxylic acid of Formula I with a compound of Formula II 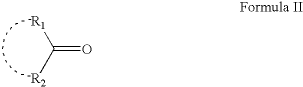
wherein R₁ and R₂ are independently hydrogen, alkyl, alicyclic, aryl, aralkyl, or R₁ and R₂ together form a 5- to 7-membered carbocyclic ring, in the presence of an acid, HX, to form a reaction mixture comprising an alkylidene ammonio salt derivative of Formula III, 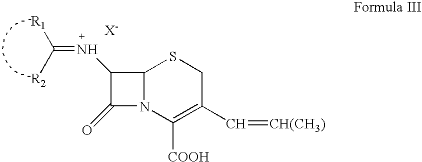
wherein R₁ and R₂ are the same as above and X⁻ is an anion from the acid HX;
(b) obtaining (Z)-isomer-enriched alkylidene ammonio salt derivative of Formula III from the reaction mixture; and
(c) converting the (Z)-isomer-enriched alkylidene ammonio salt derivative of Formula III to 7-amino-3-(1-propen-1-yl)-3-cephem-4-carboxylic acid of Formula I, as the free acid or in salt forms.

2. The process according to claim 1, wherein the compound of Formula II comprises a ketone.

3. The process according to claim 2, wherein the ketone is selected from one or more of acetone, methyl isobutyl ketone, cyclohexanone, cyclopentanone, and benzophenone.

4. The process according to claim 1, wherein the compound of Formula II comprises an aldehyde.

5. The process according to claim 4, wherein the aldehyde is selected from one or more of benzaldehyde, acetaldehyde and formaldehyde.

6. The process according to claim 1, wherein the acid comprises an inorganic acid.

7. The process according to claim 6, wherein the inorganic acid comprises one or more of hydrogen chloride, hydrogen bromide, hydrogen iodide, sulfuric acid and perchloric acid.

8. The process according to claim 1, wherein the acid comprises an organic acid.

9. The process according to claim 1, wherein the organic acid is selected from one or more of formic acid and acetic acid.

10. The process according to claim 1, wherein the reaction is performed in an inert non-aqueous organic solvent or solvent mixture in which the (Z)- and (E)-isomers of the derivative of Formula III have different solubilities.

11. The process according to claim 10, wherein the organic solvent or mixture is such that the (Z)-isomer of the salt derivative of Formula III is relatively insoluble and the (E)-isomer is soluble.

12. The process according to claim 10, wherein the organic solvent or mixture comprises one or more of carboxylic acids, amides, sulfoxides, sulfones, halogenated hydrocarbons, ketones, esters, ethers, and nitrites.

13. The process according to claim 11, wherein the organic solvent or mixture comprises one or more of acetic acid, dimethylformamide, dimethylsulfoxide, sulfolane, dichloromethane, acetone, ethyl acetate, tetrahydrofuran, and acetonitrile.

14. The process according to claim 1, wherein the reaction mixture of step (a) is diluted with a counter solvent or a mixture of counter solvents to obtain the (Z)-isomer enriched derivative of Formula III.

15. The process according to claim 14, wherein the organic counter solvent comprises one or more of ketones, ethers, esters, and nitrites.

16. The process according to claim 15, wherein the organic counter solvent comprises one or more of acetone, tertiary butyl methyl ether, diethylether, tetrahydrofuran, ethyl acetate, isopropyl acetate, and acetonitrile.

17. The process according to claim 1, wherein the reaction of step (a) is performed at a temperature of between about 20° C. to about 55° C.

18. The process according to claim 17, wherein the reaction is performed at a temperature of between about 30° C. to about 45° C.

19. The process according to claim 1, wherein obtaining the (Z)-isomer-enriched alkylidene ammonio salt derivative of Formula III comprises crystallizing the derivative of Formula III at a temperature of between about 0° C. to about 30° C.

20. The process according to claim 19, wherein the (Z)-isomer-enriched alkylidene ammonio salt derivative of Formula III is crystallized at a temperature of between about 0° C. to about 15° C.

21. The process according to claim 1, wherein conversion of the carboxylic acid of Formula I provides the compound of Formula I comprising Z/E isomers in a ratio of about 91:9 to about 99:1.

22. The process according to claim 1, further comprising converting the (Z)-isomer-enriched carboxylic acid of Formula I to a 3-propenyl cephalosporin antibiotic.

23. The process according to claim 1, further comprising converting the (Z)-isomer-enriched carboxylic acid of Formula I to cefprozil.

24. The process according to claim 23, wherein cefprozil comprises Z/E isomers in a ratio of from about 91:9 to about 99:1.

25. The process according to claim 1, further comprising obtaining cefprozil by:
silylating the (Z)-isomer enriched 7-amino-3-(1-propen-1-yl)-3-cephem-4-carboxylic acid of Formula I; and
reacting the silylated product with a mixed carboxylic acid anhydride produced by reacting a Dane salt with ethyl chloroformate.

26. A drug product comprising a 3-propenyl cephalosporin antibiotic formed by the process of claim 22.

27. A drug product comprising cefprozil formed by the process of claim 23.

28. A drug product comprising cefprozil formed by the process of claim 24.

29. A drug product comprising cefprozil formed by the process of claim 25.

30. A method of treating a condition for which an antibiotic is indicated, the method comprising providing a drug product comprising a 3-propenyl cephalosporin antibiotic formed by the process of claim 22.

31. The method of claim 30, wherein the 3-propenyl cephalosporin antibiotic comprises cefprozil.
